(19) United States
(12) Patent Application Publication
(10) Pub. No.: US 2013/0046144 A1
(43) Pub. Date: Feb. 21, 2013

(54) FLEXIBLE TUBE UNIT OF ENDOSCOPE AND ENDOSCOPE HAVING THIS FLEXIBLE TUBE UNIT

(71) Applicant: OLYMPUS MEDICAL SYSTEMS CORP, Tokyo (JP)

(72) Inventor: Taro IEDE, Hachioji-shi, (JP)

(73) Assignee: OLYMPUS MEDICAL SYSTEMS CORP, Tokyo (JP)

(21) Appl. No.: 13/656,873

(22) Filed: Oct. 22, 2012

Related U.S. Application Data
(63) Continuation of application No. PCT/JP2011/053848, filed on Feb. 22, 2011.

(30) Foreign Application Priority Data
Jun. 25, 2010 (JP) 2010-145606

(51) Int. Cl. A61B 1/01

(52) U.S. Cl. 600/140

(57) ABSTRACT

A flexible tube unit of an endoscope includes a metal spiral tube, a mesh tube which covers the metal spiral tube and is formed by braiding a wire bundle obtained by bundling wires, an envelope which covers the mesh tube and a regulating portion which regulates movement of the wires of the mesh tube. A ratio of a surface area of the regulating portion with respect to a surface area of the mesh tube differs in an axial direction of the mesh tube.

### CROSS REFERENCE TO RELATED APPLICATIONS

This application is a Continuation Application of PCT Application No. PCT/JP2011/053848, filed Feb. 22, 2011 and based upon and claiming the benefit of priority from prior Japanese Patent Application No. 2010-145606, filed Jun. 25, 2010, the entire contents of all of which are incorporated herein by reference.

### BACKGROUND OF THE INVENTION

1. Field of the Invention

The present invention relates to a thin flexible tube unit of an endoscope, which can maintain a variation width of flexibility, and to an endoscope having this flexible tube unit.

2. Description of the Related Art

In general, a flexible tube unit of an endoscope has a spiral tube, a mesh tube which is arranged outside the spiral tube and covers the spiral tube, and an envelope which is arranged outside the mesh tube and covers the mesh tube. As described above, the flexible tube unit has a three-layer structure including the spiral tube, the mesh tube, and the envelope.

A method of changing flexibility of such a flexible tube unit is disclosed in, for example, Jpn. Pat. Appln. KOKOKU Publication No. 6-98115 and Jpn. UM Appln. KOKOKU Publication No. 63-34641.

In Jpn. Pat. Appln. KOKOKU Publication No. 6-98115, as the method of changing the flexibility, an envelope is made of an elastomer, and a compounding ratio for hardness and softness of the elastomer is changed depending on a distal end portion and a proximal end portion of the flexible tube unit.

Further, in Jpn. UM Appln. KOKOKU Publication No. 63-34641, as the method of changing the flexibility, a mesh tube is coated with a synthetic resin, and a coating thickness or a material of the synthetic resin is changed depending on a distal end portion and a proximal end portion of the mesh tube.

### BRIEF SUMMARY OF THE INVENTION

According to an aspect of embodiments, a flexible tube unit of an endoscope according to the present invention comprises: a metal spiral tube; a mesh tube which covers the metal spiral tube and is formed by braiding a wire bundle obtained by bundling wires; an envelope which covers the mesh tube; and a regulating portion which regulates movement of the wires of the mesh tube, wherein a ratio of a surface area of the regulating portion with respect to a surface area of the mesh tube differs in an axial direction of the mesh tube.

According to an aspect of embodiments, a flexible tube unit of an endoscope according to the preset invention comprises: a metal spiral tube; a mesh tube which covers the metal spiral tube and is formed by braiding a wire bundle obtained by bundling wires; an envelope which covers the mesh tube; and a regulating portion which regulates movement of the mesh tube with respect to the metal spiral tube, wherein the regulating portion is arranged along an axial direction of the mesh tube.

According to an aspect of embodiments, an endoscope having the above-described flexible tube unit of an endoscope.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic block diagram of an endoscopic system according to the present invention;

FIG. 2 is a view showing a three-layer structure of a flexible tube unit;

FIG. 3 is a partially enlarged view of a mesh tube;

FIG. 4A is a schematic view showing a proximal end portion side and a distal end portion side of a mesh tube in a first embodiment, and also showing a regulating portion having a spiral shape when D1 is smaller than D2;

FIG. 4B is a schematic view showing the proximal end portion side and the distal end portion side of the mesh tube in the first embodiment, and also showing the regulating portion having a spiral shape which is discontinuous in an axial direction of the flexible tube unit when D1 is smaller than D2;

FIG. 5A is a schematic view showing a proximal end portion side and a distal end portion side of a mesh tube in a first modification of the first embodiment, and also showing a regulating portion having a spiral shape when D1 is equal to D2;

FIG. 5B is a schematic view showing the proximal end portion side and the distal end portion side of the mesh tube in the first modification of the first embodiment, and also showing a regulating portion having a spiral shape which is discontinuous in an axial direction of the flexible tube unit when D1 is equal to D2;

FIG. 6A is a view showing continuous ring-shaped regulating portions when D1 is smaller than D2;

FIG. 6B is a view showing ring-shaped regulating portions which are discontinuous in a circumferential direction of the flexible tube unit when D1 is smaller than D2;

FIG. 7A is a view showing continuous ring-shaped regulating portions when D1 is equal to D2; and

FIG. 7B is a view showing ring-shaped regulating portions which are discontinuous in the circumferential direction of the flexible tube unit when D1 is equal to D2.

### DETAILED DESCRIPTION OF THE INVENTION

Hereinafter, embodiments according to the present invention will be described with reference to the accompanying drawings

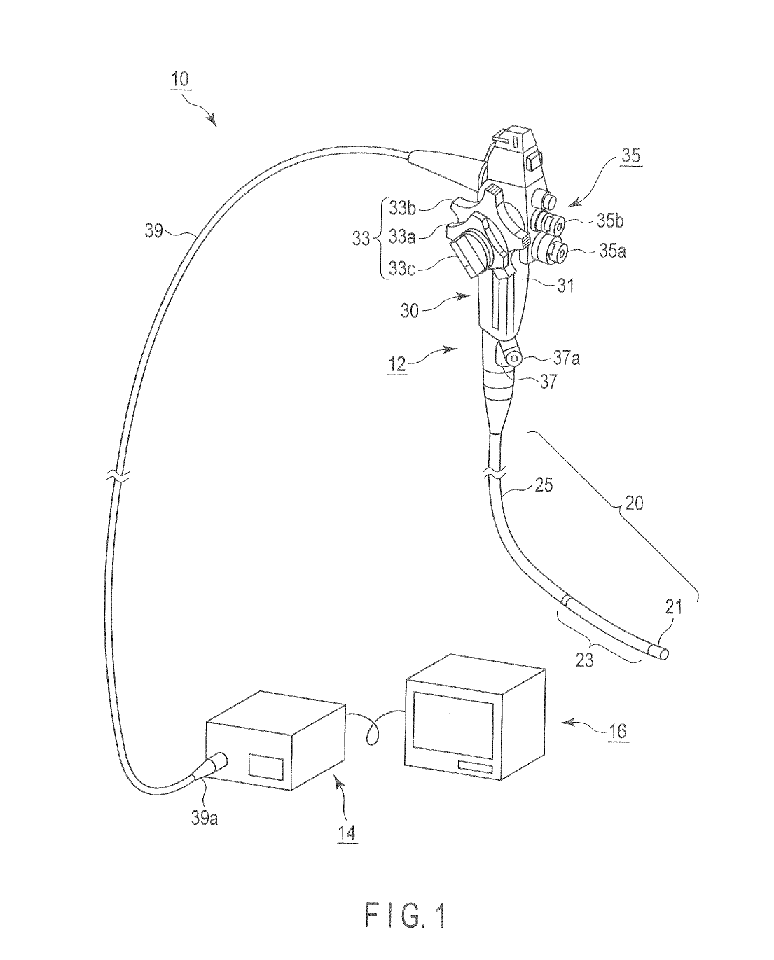

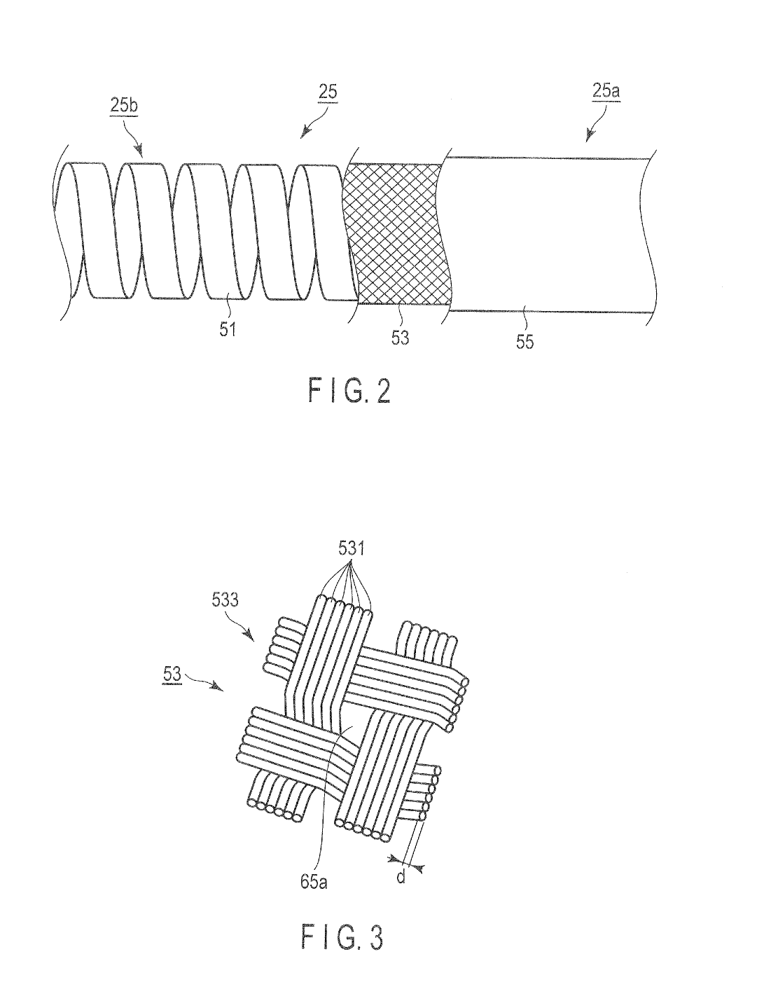

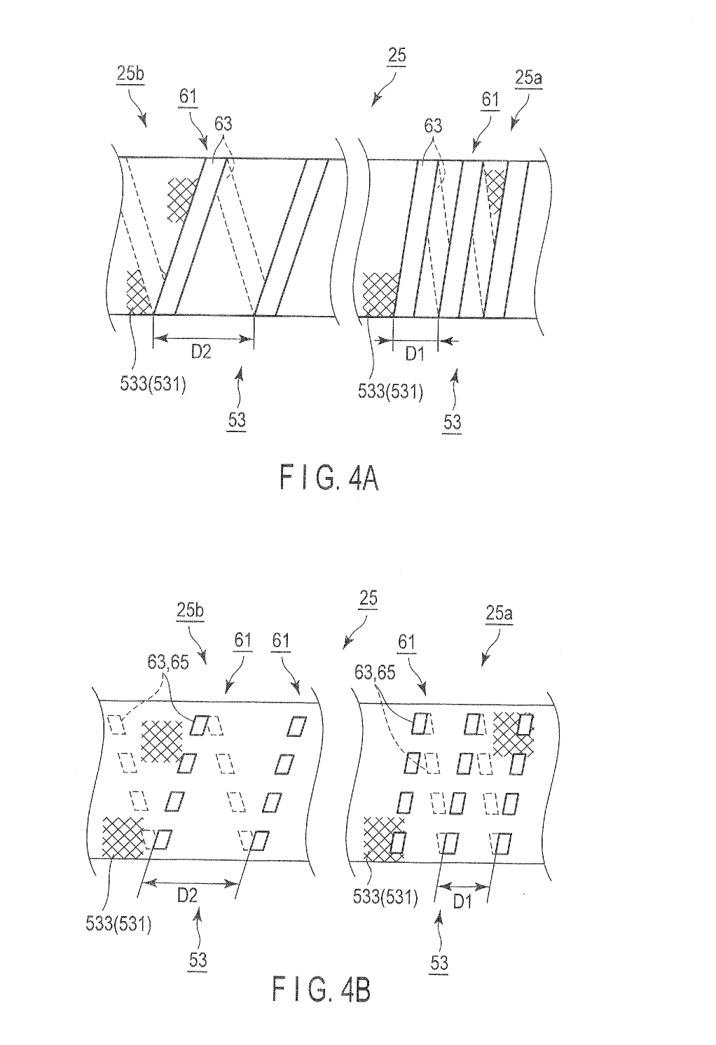

A first embodiment will now be described hereinafter with reference to FIG. 1, FIG. 2, FIG. 3, and FIG. 4A.

As shown in FIG. 1, an endoscopic system 10 has an endoscope 12 that images, for example, a desired observation target, an image processing device 14 (for example, a video processor) that is detachably connected to the endoscope 12, and a monitor 16 which is a display unit that is connected to the image processing device 14 and displays the observation target imaged by the endoscope 12. This observation target means, for example, an affected part or a diseased part in a subject (for example, a body cavity).

The endoscope 12 has an elongated insertion unit 20 which is inserted into the subject and an operation unit 30 which is arranged at a proximal end portion of this insertion unit 20 and operates the insertion unit 20.

The insertion unit 20 has a distal end hard unit 21, a bending unit 23, and a flexible tube unit 25 from a distal end portion side of the insertion unit 20 toward a proximal end portion side of the insertion unit 20. A proximal end portion of the distal end hard unit 21 is coupled with a distal end portion of the bending unit 23, and a proximal end portion of the bending unit 23 is coupled with a distal end portion 25b of the flexible tube unit 25.

The distal end hard unit 21 is a distal end portion of the insertion unit 20.

The bending unit 23 is connected to a later-described bending operation unit 33 of the operation unit 30 through an operation wire (not shown) inserted in the flexible tube unit 25. The bending unit 23 is bent in desired directions, for example, up, down, left, and right directions by an operation of the bending operation unit 33. When the bending unit 23 bends, a position and a direction of the distal end hard unit 21 change, the observation target is captured in an observation visual field (or an imaging viewing field), and illumination light is applied to the observation target.

It is to be noted that the bending portion 23 is constituted by aligning non-illustrated substantially cylindrical (annular) node rings along a longitudinal axis direction of the insertion unit 20 (an inserting direction of the endoscope 12). The node rings adjacent to each other (sequentially placed along the longitudinal axis direction of the insertion unit 20) are coupled with each other through a pivot (a spindle portion) such as a rivet to allow their rotational movement. When the node rings are coupled to allow their rotational movement in this manner, the bendable (turnable) bending unit 23 is formed as described above. It is to be noted that a non-illustrated node ring which is the closest to the distal end hard unit 21 is coupled with the distal end hard unit 21.

The distal end hard unit 21 and the bending unit 23 are covered with a non-illustrated envelope tube. This envelope tube is made of, for example, a resin material such as rubber or an elastic material. Further, the envelope tube is formed into substantially the same shape (for example, a hollow shape or a cylindrical shape) as the distal end hard unit 21 and the bending unit 23. It is to be noted that the envelope tube may be injection-molded by using an elastic material which is a material of a thermoplastic elastomer (for example, a styrene base, an olefin base, or an urethane base). It is to be noted that molding of the thermoplastic elastomer is not restricted to the injection molding, and various molding methods such as cast molding, extrusion, blow molding may be adopted.

The flexible tube unit 25 is a tubular member which has desired flexibility and is extended from the operation unit 30, and it is bent by external force. A detailed configuration of the flexible tube unit 25 will be described later.

The operation unit 30 has an operation unit main body 31 which is a grip portion that grips the endoscope 12 and a universal cord 39 which is connected to the operation unit main body

A bending operation unit 33 that bends the bending unit 23 is arranged in the operation unit main body 31. The bending operation unit 33 has a left-and-right bending operation knob 33a that bends the bending unit 23 in left and right directions, an up-and-down bending operation knob 33b which bends the bending operation unit 23 in up and down directions, and a fixing knob 33c that fixes a position of the bent bending unit 23.

A non-illustrated up-and-down direction bending operation mechanism which is driven by the left-and-right bending operation knob 33a is connected to the left-and-right bending operation knob 33a. Further, a non-illustrated up-and-down direction bending operation mechanism which is driven by the up-and-down bending operation knob 33b is connected to the up-and-down bending operation knob 33b. The up-and-down direction bending operation mechanism and the left-and-right direction bending operation mechanism are arranged in the operation unit 30.

The left-and-right direction bending operation mechanism is connected to a non-illustrated operation wire which is inserted into the flexible tube unit 25 and the bending unit 23, and this operation wire is connected to the bending unit 23.

Furthermore, the up-and-down direction bending operation mechanism is connected to a non-illustrated operation wire which is inserted into the bending tube unit 25 and the bending unit 23. The operation wire connected with the up-and-down direction bending operation mechanism is different from the operation wire connected with the left-and-right direction bending operation mechanism. The operation wire connected with the up-and-down direction bending operation mechanism is connected with the bending unit 23.

The left-and-right bending operation knob 33a bends the bending unit 23 in the left and right directions through the left-and-right direction bending operation mechanism and the operation wire. Additionally, the up-and-down bending operation knob 33b bends the bending unit 23 in the up and down directions through the up-and-down direction bending operation mechanism and the operation wire.

Further, a switch unit 35 is arranged on the operation unit main body 31. The switch unit 35 has a suction switch 35a and an air supply/water supply switch 35b. The switch unit 35 is operated by an operator's hand when the operation unit main body 31 is held by the operator. The suction switch 35a is operated when the endoscope 12 sucks a mucosal fluid from the distal end hard unit 21. The air supply/water supply switch 35b is operated when the endoscope 12 is operated for air supply/water supply in order to secure mainly a clean observation viewing field in the distal end hard unit 21.

Furthermore, a surgical instrument insertion unit 37 is arranged on the operation unit main body 31. A surgical instrument insertion opening 37a is arranged in the surgical instrument insertion unit 37. A proximal end portion of a non-illustrated surgical instrument insertion channel arranged from the flexible tube unit 25 to the distal end hard unit 21 in the insertion unit 20 is coupled with the surgical instrument insertion opening 37a. The surgical instrument insertion opening 37a is an insertion opening from which a non-illustrated endoscopic surgical instrument is inserted into the surgical instrument insertion channel. The non-illustrated endoscopic surgical instrument is inserted into the surgical instrument insertion channel from the surgical instrument insertion opening 37a. The non-illustrated endoscopic surgical instrument is pressed into the distal end hard unit 21 side and then protruded from a non-illustrated distal end opening portion of the surgical instrument insertion channel arranged in the distal end hard unit 21.

The universal cord 39 is extended from a side surface of the operation unit main body 31. The universal cord 39 has a connector 39a, which is attachable to or detachable from the image processing device 14, at an end portion thereof.

A configuration of the flexible tube unit 25 according to this embodiment will now be described hereinafter in detail with reference to FIG. 2, FIG. 3, and FIG. 4A.

The flexible tube unit 25 has, for example, a hollow shape. In detail, as shown in FIG. 2, the flexible tube unit 25 has a spiral tube 51, a mesh tube 53 which is arranged outside this spiral tube 51 and covers the spiral tube 51, and an envelope 55 which is arranged outside this mesh tube 53 and covers the mesh tube 53. The mesh tube 53 laminated on the spiral tube 51, and the spiral tube 51 laminated on the envelope 55.

The spiral tube 51 is formed into a substantially cylindrical shape by forming a strip-like thin plate material made of, for example, stainless steel into a spiral shape. The spiral tube 51 is, for example, a thin-wail metal spiral tube.

As shown in FIG. 2 and FIG. 3, the mesh tube 53 is formed by braiding each wire bundle 533 obtained by bundling wires 531 made of, for example, stainless steel into a substantially cylindrical shape. In the mesh tube 53, the wire bundles 533 are crossed to form a mesh pattern. A thickness of this mesh tube 53 corresponds to a sum of outside diameters of the two wires 531. For example, when an outside diameter of one wire 531 is d, a thickness of the mesh tube 53 is 2d. In this embodiment, d is, for example, 0.08 or 1.12 mm.

The envelope 55 is formed into a substantially cylindrical shape by using a resin material having flexibility such as a rubber material to cover the outer side of the mesh tube 53.

The flexible tube unit 25 has a three-layer structure including the spiral tube 51, the mesh tube 53, and the envelope 55 described above.

In this embodiment, to improve insertability of the endoscope 12 into a body cavity, the flexible tube unit 25 gradually becomes softer from the proximal end portion 25a side of the flexible tube unit 25 toward the distal end portion 25b side of the flexible tube unit 25. The proximal end portion 25a side is harder than the distal end portion 25b side. Furthermore, the flexible tube unit 25 must maintain the flexibility and a variation width of the flexibility must be maintained. The variation width of the flexibility means a difference in hardness between the distal end portion 25b and the proximal end portion 25a.

Therefore, the flexibility of the mesh tube 53 differs in the axial direction of the mesh tube 53. In more detail, when the mesh tube 53 is formed by braiding each wire bundle 533 obtained by bundling the wires 531, as shown in FIG. 4A, the mesh tube 53 has a regulating portion 61 formed by joining the wires 531. At this time, a ratio of a surface area of the regulating portion 61 with respect to a surface area of the mesh tube 53 differs in the axial direction of the flexible tube unit 25 (the mesh tube 53), namely, depending on the proximal end portion 25a side of the flexible tube unit 25 and the distal end portion 25b side of the flexible tube unit 25. That is, in the flexibility of the mesh tube 53, the distal end portion 25b side is apt to bend as compared with the proximal end portion 25a side. In more detail, the flexible tube unit 25 gradually becomes soft from the proximal end portion 25a side toward the distal end portion 25b side. Therefore, the regulating portion 61 is formed in such a manner that the ratio of the surface area of the regulating portion 61 with respect to the surface area of the mesh tube 53 on the proximal end portion 25a side is higher than the ratio of the surface area of the regulating portion 61 with respect to the surface area of the mesh tube 53 on the distal end portion 25b side.

The regulating portion 61 is formed by joining the wires 531 forming the wire bundle 533 and regulates movement of the wires 531 of the mesh tube 53. Additionally, the regulating portion 61 regulates the movement of the mesh tube 53 with respect to the spiral tube 51.

As shown in FIG. 4A, the regulating portion 61 is formed into a spiral shape with respect to the axial direction of the flexible tube unit 25 like the spiral tube 51. Such a regulating portion 61 is arranged along the axial direction of the mesh tube 53. The regulating portion 61 is formed by joining the wires 531 by, for example, a laser or soldering.

That is, as shown in FIG. 4A, the regulating portion 61 does not cover the mesh tube 53, but it joins some of the wires 533 forming the mesh tube 53 by a laser or soldering and functions as a part of the mesh tube 53. Therefore, in the mesh tube 53 excluding the regulating portion 61, the wire bundle 533 obtained by bundling the wires 531 is braided into a substantially mesh pattern.

As shown in FIG. 4A, the regulating portion 61 has a regulating band portion 63 serving as one band which is obtained by joining the wires 531 through a laser or soldering in the wire bundle 533 and forming them into a spiral shape in the axial direction of the flexible tube unit 25. Therefore, as shown in FIG. 4A, the regulating band portion 63 is formed as one spiral metal wire without being disconnected.

For example, the number of the wires 531 joined together in the regulating band portion 63 on the proximal end portion 25a side is the same as the number of the wires 531 joined together in the regulating band portion 63 on the distal end portion 25b side. In this case, the number of the wires 531 joined together is four, whereby a thickness of the regulating band portion 63 on the proximal end portion 25a side is equal to that on the distal end portion 25b side. Further, in this case, as shown in FIG. 4A, intervals D1 and D2 (pitches) between parts of the regulating hand portion 63 in the axial direction of the flexible tube unit 25 vary in the axial direction of the flexible tube unit 25. In detail, intervals D1 and D2 differ depending on the proximal end portion 25a side and the distal end portion 25b side. That is, a ratio of the surface area of the regulating band portion 63 with respect to the surface area of the mesh tube 53 differs depending on the proximal end portion 25a side and the distal end portion 25b side.

In more detail, as shown in FIG. 4A, interval D1 between parts of the regulating band portion 63 on the proximal end portion 25a side is smaller than interval D2 between parts of the regulating band portion 63 on the distal end portion 25b side. As a result, the ratio of the surface area of the regulating band portion 63 with respect to the surface area of the mesh tube 53 on the proximal end portion 25a side is higher than the ratio of the surface area of the regulating band portion 63 with respect to the surface area of the mesh tube 53 on the distal end portion 25b side, and the flexible tube unit 25 gradually becomes softer from the proximal end portion 25a side toward the distal end portion 25b side. That is, the flexibility of the mesh tube 53 is dependent on the ratio of the surface area of the regulating band portion 63 with respect to the surface area of the mesh tube 53.

In other words, although the thickness of the regulating band portion 63 is the same on both the proximal end portion a side and the distal end portion 25b side, the interval of the regulating band portion 63 is narrowed from the distal end portion 25b side toward the proximal end portion 25a side. Therefore, the regulating band portion 63 is arranged to be dense from the distal end portion 25b side toward the proximal end portion 25a side.

As a result, more regulating wires 531 are arranged on the proximal end portion 25a side than the distal end portion 25b side, and movement is restricted more on the proximal end portion 25a side than the distal end portion 25b side. Therefore, the proximal end portion 25a side becomes harder than the distal end portion 25b side, whereby the proximal end portion 25a side becomes a hard portion and the distal end portion 25b side becomes a soft portion. Further, the flexible tube unit 25 gradually becomes softer from the proximal end portion 25a side toward the distal end portion 25b side, and the flexible tube unit 25 bends from the distal end portion 25b side.

An operating method according to this embodiment will now be described will now be described.

As shown in FIG. 3, each wire bundle 533 obtained by braiding the wires 531 made of, for example, stainless steel into a substantially cylindrical shape, whereby the mesh tube 53 is formed. At this time, the wire bundles 533 are crossed each other in a mesh pattern.

In the mesh tube 53, when some of the wire bundles 533 are joined together by a laser or soldering, such a regulating portion 61 as shown in FIG. 4A is formed. At this time, in the regulating portion 61 according to this embodiment, as shown in FIG. 4A, the regulating band portion 63 in which the wires 531 are joined together by, for example, a laser or soldering is formed. As shown in FIG. 4A, the regulating band portion 63 serves as one band, and it is formed into a spiral shape along the axial direction of the flexible tube unit 25.

In this embodiment, the number of the wires 531 joined together in the regulating band portion 63 on the proximal end portion 25a side is determined to be equal to the number of the wires 531 joined together in the regulating band portion 63 on the distal end portion 25b side. Furthermore, as shown in FIG. 4A, interval D1 is smaller than interval D2. As a result, the ratio of the surface area of the regulating hang portion 63 with respect to the surface area of the mesh tube 53 on the proximal end portion 25a side is higher than the ratio of the surface area of the regulating band portion 63 with respect to the surface area of the mesh tube 53 on the distal end portion 25b side. Therefore, movement on the proximal end portion 25a side is more restricted than on the distal end portion 25b side, and the proximal end portion 25a side becomes harder than the distal end portion 25b side.

When the insertion unit 20 is inserted into a body cavity, since the proximal end portion 25a side is harder than the distal end portion 25b side, the flexible tube unit 25 bends from the distal end portion 25b side.

The flexibility of the flexible tube unit 25 according to this embodiment is determined by the ratio of the surface area of the restricting band portion 63 with respect to the surface area of the mesh tube 53, and it is not dependent on the envelope 55. Therefore, even if the endoscope 12 is used for a long time or repeatedly used or the envelope 55 is deteriorated due to a medical agent used for cleaning/sterilization, a change in flexibility of the flexible tube unit 25 is slight, and an initial variation width of the flexibility is maintained.

Additionally, since the mesh tube 53 alone is processed, the flexible tube unit 25 does not have a large diameter.

As described above, in this embodiment, even if prolonged use or repeated use is effected, the variation width of the flexibility in the initial state indicative of characteristics of the flexible tube unit 25 can be maintained without being dependent on the envelope 55.

Further, in this embodiment, since only the mesh tube 53 is processed, even if the flexible tube unit 25 is used for a long time or repeatedly used irrespective of deterioration of the envelope 55, the regulating band portion 63 is not deteriorated. Therefore, in this embodiment, a value of the flexibility is not lowed due to an aging variation, the initial state can be maintained, a variation width of the flexibility the initial state can be maintained, and a decrease in insertability of the endoscope 12 into a body cavity can be avoided.

Moreover, in this embodiment, to maintain the variation width of the flexibility, a thickness of envelope 55 must not to be increased, and a thickness of the flexible tube unit 25 is decreased.

Moreover, in this embodiment, to change the flexibility, the metal mesh tube 53 is processed without changing a coating thickness of the synthetic resin of the envelope 55. As a result, in this embodiment, a desired variation width of the flexibility can be given to the flexible tube unit 25 without increasing the diameter of the flexible tube unit 25.

Additionally, in this embodiment, the wires 531 are joined together in such a manner that the ratio of the surface area of the regulating band portion 63 with respect to the surface area of the mesh tube 53 on the proximal end portion 25a side can be higher than the ratio of the surface area of the regulating band portion 63 with respect to the surface area of the mesh tube 53 on the distal end portion 25b side, thereby regulating movement on the proximal end portion 25a side more than on the distal end portion 25b side. Therefore, in this embodiment, hardness can be changed depending on the distal end portion 25b side and the proximal end portion 25a side, thereby maintaining the variation width of the flexibility.

Further, in this embodiment, when the regulating portion 61 is formed into a spiral shape, collapse resisting properties of the flexible tube unit 25 in the radial direction can be improved.

It is to be noted that, in this embodiment, the regulating band portion 63 may be discontinuous as long as it is formed into the spiral shape as shown in FIG. 4B. That is, the regulating portion 61 may have regulating parts 65 where the wires 531 are spirally formed in the wire bundle 533 in the axial direction of the flexible tube unit 25 by partially joining using, for example, a laser or soldering.

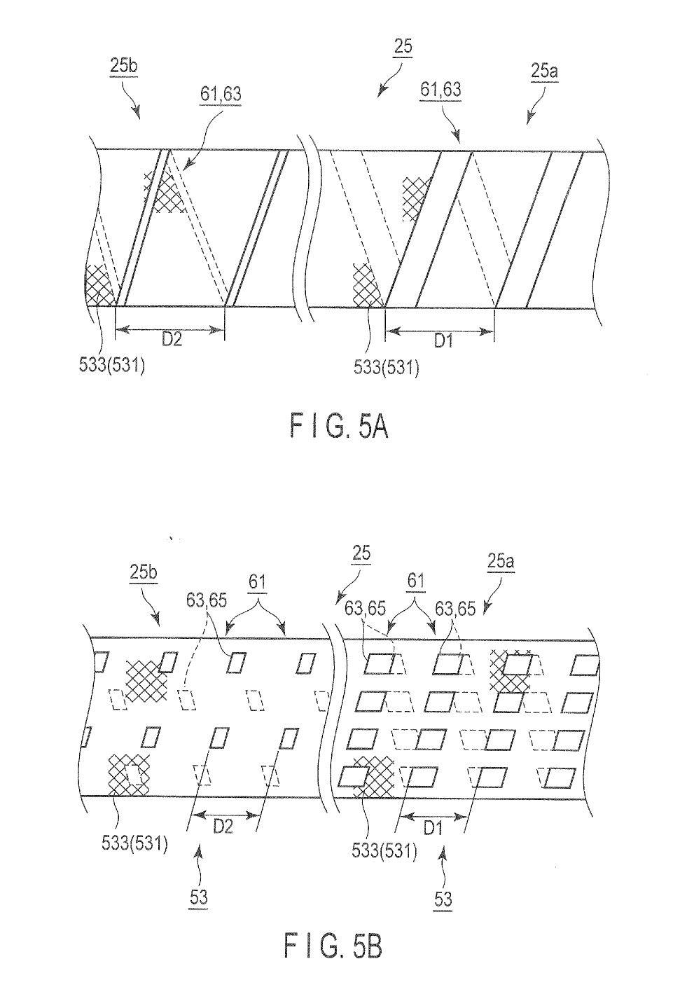

A first modification of this embodiment will now be described wide reference to FIG. 5A.

In this modification, when interval D1 on the proximal end portion 25a side is equal to interval D2 on the distal end portion 25b side as shown in FIG. 5A, the number of the wires 531 joined together in the regulating band portion 63 differs depending on the proximal end portion 25a side and the distal end portion 25b side as shown in FIG. 5A. That is, on the proximal end portion 25a side and the distal end portion 25b side, the ratio of the surface area of the regulating band portion 63 with respect to the surface area of the mesh tube 53, namely, a width of the regulating portion 61 changes in the axial direction of the flexible tube unit 25 and, to be exact, this width differs.

In other words, in the regulating portion 61 having the uniform intervals, since the regulating band portion 63 gradually becomes thicker from the distal end portion 25b side toward the proximal end portion 25a side, that is, the number of the wires 531 joined together is increased from the distal end portion 25b side toward the proximal end portion 25a side, the regulating band portion 63 is arranged to be dense from the distal end portion 25b side toward the proximal end portion 25a side.

As a result, more joined wires 531 are arranged on the proximal end portion 25a side than the distal end portion 25b side, and movement on the proximal end portion 25a side is more restricted than on the distal end portion 25b side. Consequently, the proximal end portion 25a side becomes harder than the distal end portion 25b side, and the flexible tube unit 25 bends from the distal end portion 25b side in a state that the variation width of the flexibility is maintained.

As described above, in this modification, the same effects as those of the first embodiment can be obtained.

It is to be noted that, in this modification, like FIG. 4B, the regulating band portion 63 may be discontinuous as long as it is formed into a spiral shape as shown in FIG. 5B. That is, in the wire bundles 533, the regulating portion 61 may have the regulating parts 65 where the wires 531 are formed into the spiral shape in the axial direction of the flexible tube unit 25 by partially joining using, for example, a laser or joining.

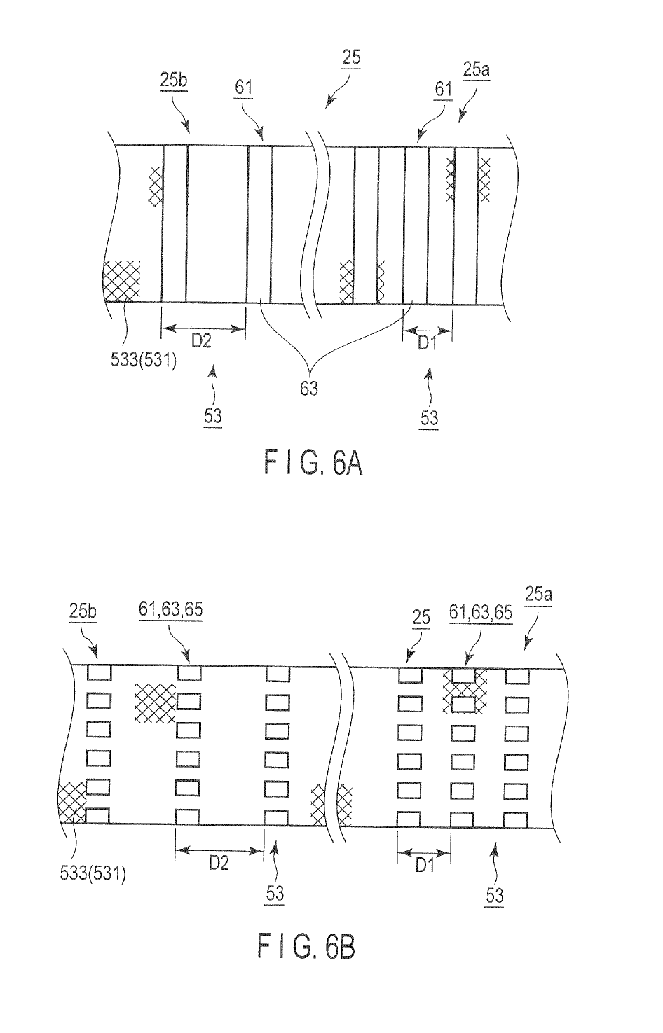

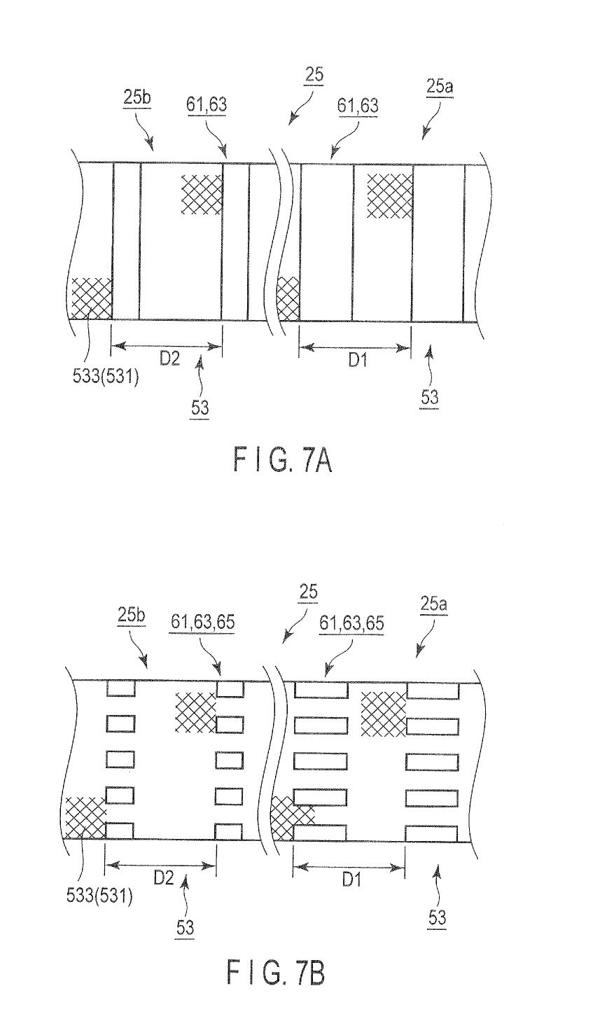

It is to be noted that the regulating portion 61 has the spiral shape in the first embodiment and the modification thereof, but the present invention is not restricted thereto, and the regulating portion 61 may have a ring-like shape in the circumferential direction of the flexible tube unit 25 as shown in FIG. 6A, FIG. 6B, FIG. 7A, and FIG. 7B.

At this time, the respective regulating portions 61 are not continuous, and they are separated from each other.

For example, as shown in FIG. 6A, D1 is smaller than D2 like FIG. 4A, and the regulating portion 63 is continuous in the circumferential direction of the flexible tube unit 25 and has a ring-like shape. Therefore, a ratio of the surface area of the regulating portion 61 with respect to the surface area of the mesh tube 53 on the proximal end portion 25a side is higher than a ratio of the surface area of the regulating portion 61 with respect to the surface area of the mesh tube 53 on the distal end portion 25b side.

Furthermore, for example, as shown in FIG. 6B, D1 is smaller than D2 like FIG. 4B, and the regulating band portion 63 may be discontinuous as long as it is formed into a ring-like shape in the circumferential direction of the flexible tube unit 25. That is, in the wire bundles 533, the regulating portion 61 has regulating portions 65 where the wires 531 are partially formed in the circumferential direction of the flexible tube unit 25 by joining using, for example, a laser or soldering.

Moreover, for example, as shown in FIG. 7A, like FIG. 5A, D1 is equal to D2, the regulating band portion 63 is continuous in the circumferential direction of the flexible tube unit 25 and has the ring-like shape. At this time, in the regulating portion 61 having uniform intervals, the regulating band portions 63 gradually become thicker from the distal end portion 25b side toward the proximal end portion 25a side.

Additionally, for example, as shown in FIG. 7B, like FIG. 5B, D1 is equal to D2, and the regulating band portion 63 may be discontinuous as long as it is formed into the ring-like shape in the circumferential direction of the flexible tube unit 25. That is, in the wire bundles 533, the regulating portion 61 has the regulating portions 65 where the wires 531 are partially formed in the circumferential direction of the flexible tube unit 25 by joining using, for example, a laser or soldering.

In this embodiment, when each regulating portion 61 is formed into a ring-like shape, like the example of the spiral shape, the collapse resisting properties of the flexible tube unit 25 in the radial direction can be improved.

The present invention is not restricted to the foregoing embodiment as it is, and it can be embodied by modifying constituent elements without departing from the gist on the embodying stage. Additionally, appropriately combining the constituent elements disclosed in the foregoing embodiment enables forming the various inventions.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A flexible tube unit of an endoscope, comprising:
a metal spiral tube;
a mesh tube which covers the metal spiral tube and is formed by braiding a wire bundle obtained by bundling wires;
an envelope which covers the mesh tube; and
a regulating portion which regulates movement of the wires of the mesh tube,
wherein a ratio of a surface area of the regulating portion with respect to a surface area of the mesh tube differs in an axial direction of the mesh tube.

2. The flexible tube unit of an endoscope according to claim 1,
wherein a distal end portion side of the mesh tube is apt to bend as compared with a proximal end portion side of the mesh tube.

3. The flexible tube unit of an endoscope according to claim 1,
wherein the regulating portion is formed into a spiral shape with respect to the axial direction.

4. The flexible tube unit of an endoscope according to claim 3,
wherein a pitch in the regulating portion varies in the axial direction.

5. The flexible tube unit of an endoscope according to claim 3,
wherein a width of the regulating portion varies in the axial direction.

6. The flexible tube unit of an endoscope according to claim 1,
wherein the regulating portion has a ring-like shape in a circumferential direction of the mesh tube.

7. The flexible tube unit of an endoscope according to claim 6,
wherein a pitch in the regulating portion varies in the axial direction.

8. The flexible tube unit of an endoscope according to claim 6,
wherein a width of the regulating portion varies in the axial direction.

9. The flexible tube unit of an endoscope according to claim 1,
wherein the regulating portion is formed by joining the wires forming the wire bundle.

10. The flexible tube unit of an endoscope according to claim 9,
wherein the regulating portion is formed by joining the wires by a laser or soldering.

11. An endoscope comprising the flexible tube unit of the endoscope according to claim 1.

12. A flexible tube unit of an endoscope, comprising:
a metal spiral tube;
a mesh tube which covers the metal spiral tube and is formed by braiding a wire bundle obtained by bundling wires;
an envelope which covers the mesh tube; and
a regulating portion which regulates movement of the mesh tube with respect to the metal spiral tube,
wherein the regulating portion is arranged along an axial direction of the mesh tube.

13. The flexible tube unit of an endoscope according to claim 12,
wherein a distal end portion side of the mesh tube is apt to bend as compared with a proximal end portion side of the mesh tube.

14. The flexible tube unit of an endoscope according to claim 12,
wherein the regulating portion is formed into a spiral shape with respect to the axial direction.

15. The flexible tube unit of an endoscope according to claim 14,
wherein a pitch in the regulating portion varies in the axial direction.

16. The flexible tube unit of an endoscope according to claim 14,
wherein a width of the regulating portion varies in the axial direction.

17. The flexible tube unit of an endoscope according to claim 12,
wherein the regulating portion has a ring-like shape in a circumferential direction of the mesh tube.

18. The flexible tube unit of an endoscope according to claim 17,
wherein a pitch in the regulating portion varies in the axial direction.

19. The flexible tube unit of an endoscope according to claim 17,
wherein a width of the regulating portion varies in the axial direction.

20. The flexible tube unit of an endoscope according to claim 12,
wherein the regulating portion is formed by joining the wires forming the wire bundle.

21. The flexible tube unit of an endoscope according to claim 20,
wherein the regulating portion is formed by joining the wires by a laser or soldering.

22. An endoscope comprising the flexible tube unit of the endoscope according to claim 12.
